(19) United States
(12) Patent Application Publication
(10) Pub. No.: US 2016/0161433 A1
(43) Pub. Date: Jun. 9, 2016

(54) Electrospun Polymer Fibers for Gas Sensing

(71) Applicant: Massachusetts Institute of Technology, Cambridge, MA (US)

(72) Inventors: Gregory C. Rutledge, Newton, MA (US); Yuxi Zhang, Peerland, TX (US); Harry L. Tuller, Wellesley, MA (US)

(21) Appl. No.: 14/714,785

(22) Filed: May 18, 2015

Related U.S. Application Data
(60) Provisional application No. 61/994,481, filed on May 16, 2014.

(51) Int. Cl. G01N 27/12; G01N 33/00

(52) U.S. Cl. CPC G01N 27/126; G01N 33/004; G01N 33/0039; G01N 33/0044; G01N 33/0042; G01N 33/0036; G01N 33/005; G01N 27/128

(57) ABSTRACT

Disclosed herein are fibers made from intrinsically conductive polymers, such as polyaniline, that are useful as chemiresistive gas sensors. The experimental results, based on both sensitivity and response time, show that doped PAni fibers are excellent ammonia sensors. and undoped PAni fibers are excellent nitrogen dioxide sensors.

### RELATED APPLICATIONS

This application claims the benefit of priority to U.S. Provisional Patent Application Ser. No. 61/994,481, filed May 16, 2014, the contents of which are hereby incorporated by reference.

### GOVERNMENT SUPPORT

This invention was made with Government support under Grant No. W911NF-070D-0004 awarded by the U.S. Army. The government has certain rights in this invention.

### BACKGROUND

Several recent studies have reported the development of different types of gas sensors in which nanofibers or nanowires are used to detect trace amounts of harmful gases effectively and rapidly. In particular, electrically conductive polymer nanofibers have been suggested to be promising candidates as chemiresistive sensor materials. The unique combination of high specific surface area, mechanical flexibility, room temperature operation, low cost of fabrication, and large range of conductivity change makes these materials particularly attractive as nanoscale resistance-based sensors.

Electrospinning is a convenient method to produce polymer nanofibers with diameters on the order of tens of nanometers to microns. The resulting nonwoven fiber mats have high specific surface areas, around 1 to 100 m²/g, compared to films and conventional fibers. Intrinsically conducting polymers (ICPs), such as polyaniline (PAni) doped with (+)-camphor-10-sulfonic acid (HCSA), are particularly suited to the application of gas sensing because of the ease with which its conductivity is modified. The activity of the dopant can be switched reversibly between oxidation and reduction states simply by exposure to acidic and basic gases, respectively. However, PAni is relatively hard to process into fibers, compared to most other polymers, due to its rigid backbone and relatively low molecular weight, which leads to solutions with only modest elasticity. The elastic component of the viscoelastic solution behavior has been shown to be crucial to the formation of uniform fibers in electrospinning.

Continuous fibers of pure PAni doped with HCSA have been produced. These fibers were shown to exhibit electrical conductivities as high as 130 S/cm when fully doped, and thus present a broader range of tunable conductivity with which to work during gas sensing than most of the similar systems reported to date.

There exists a need for a method of sensing gases using polymeric nanofibers.

### SUMMARY

In certain embodiments, the invention relates to a fiber consisting essentially of a polymer selected from the group consisting of: a polyacetylene, a polypyrrole, a polythiophene, and a poly(3,4-ethylenedioxythiophene) (PEDOT).

In certain embodiments, the invention relates to a fiber consisting essentially of a dopant and a polymer, wherein the polymer is selected from the group consisting of: a polyacetylene, a polypyrrole, a polythiophene, and a poly(3,4-ethylenedioxythiophene) (PEDOT).

In certain embodiments, the invention relates to any one of the fibers described herein, wherein the fiber has at least one dimension, e.g., a width or diameter, of about 1 nm to about 1 μm.

In certain embodiments, the invention relates to a sensor comprising a plurality of fibers described herein configured as a non-woven material.

In certain embodiments, the invention relates to a gas-sensing device comprising a plurality of fibers described herein.

In certain embodiments, the invention relates to a method of detecting a gas in a sample, comprising the steps of:

optionally determining the electrical resistance (R₀) or electrical conductance of a fiber;

contacting with the fiber a quantity of the sample; and

after a period of time, determining the electrical resistance (Rₑₓ) or electrical conductance of the fiber.

In certain embodiments, the invention relates to a method of detecting and quantifying a gas in a sample, comprising the steps of:

(a) optionally determining the electrical resistance (R₀) or electrical conductance of a fiber;

(b) contacting with the fiber a first standard sample, wherein the concentration of the gas in the first standard sample is known;

(c) after a period of time, determining the electrical resistance or electrical conductance of the fiber;

(d) contacting with the fiber a second standard sample, wherein the concentration of the gas in the second standard sample is known; and the concentration of the gas in the second standard sample is different from the concentration of the gas in the first standard sample;

(e) after a period of time, determining the electrical resistance or electrical conductance of the fiber;

(f) contacting with the fiber a quantity of the sample; and

(g) after a period of time, determining the electrical resistance (Rₑₓ) or electrical conductance of the fiber.

In certain embodiments, the invention relates to any one of the aforementioned methods, wherein the fiber is any one of the fibers described herein.

### BRIEF DESCRIPTION OF THE FIGURES

FIG. 1 depicts an illustration of a gas sensing apparatus, including the tube furnace, the location of the interdigitated electrodes (IDE) (zoomed in), the mass flow controllers (MFC), the computer for LabView control and data collection and analyzer interface.

FIG. 2 depicts SEM images of electrospun PAni/HCSA fibers with different molar ratios of HCSA to PAni: [HCSA]/[PAni]=0 (a); 0.5 (b); 0.75 (c); and 1.0 (d). All images taken after dissolution of the PMMA shell, and using 7,500× magnification (scale bar=2 μm).

FIG. 3 depicts the time response (solid curve) of a drawn PAni fiber (d=450 nm) with mole ratio [HCSA]/[PAni]=1.0 under cyclic exposure to 500 ppm of ammonia; dashed line indicates the cycling of the ammonia concentration between 0 and 500 ppm.

FIG. 4 depicts the time response of (a) as-spun doped PAni fibers (d=620 nm) and (b) solid-state drawn doped PAni fibers (d=450 nm), to different concentrations of NH₃; dashed lines show the change in NH₃ concentration in the test gas. The mole ratio of [HCSA]/[PAni] is 1.0.

FIG. 5 depicts changes in the resistance upon exposure to different concentrations of ammonia gas of electrospun PAni fibers with mole ratio [HCSA]/[PAni]=1.0: as-spun fibers (d=620 nm) (filled diamonds); solid-state drawn fibers (d=450 nm) (filled triangles); and lines with best polynomial fits to the data.

FIG. 6 depicts the time response (solid circles) of as-spun undoped PAni fibers (d=650 nm) under cyclic exposure to increasing concentrations of NO₂ (dashed line).

FIG. 7 depicts changes in the resistance upon exposure to different concentrations of NO₂ gas of as-spun undoped electrospun PAni fiber (d=650 nm).

FIG. 8 depicts the results of a reaction-diffusion model showing the ratio of resistances prior to and after exposure, plotted as a functions of Damköhler number (Da) and dimensionless time (τ) for selected values of equilibrium constant (K). Calculations assume as the initial condition that no gaseous reactant or product is present in the fibers, and that the conductivity of the fiber decreases linearly with the concentration of the reactant Φ.

FIG. 9 depicts the equilibrium fractions of dopant (after partial de-doping by the gas) in fibers of doped PAni calculated based on sensing responses at different ammonia concentrations. As-spun fibers (d=620 nm) (filled diamonds); solid-state drawn fibers (d=450 nm) (filled triangles).

FIG. 10 depicts a comparison of experimental data (markers) and fitted values (solid and dotted lines) for as-spun and solid-drawn doped PAni fibers upon exposure to ammonia at concentrations ranging from 10 to 700 ppm.

FIG. 11 depicts a comparison of experimental data (markers) and fitted values (solid lines) for three sensing response time series: at external ammonia concentrations of 20 ppm (filled diamonds); 100 ppm (filled squares); and 500 ppm (filled triangles).

FIG. 12 depicts the results of a reaction-diffusion model at K=30 based on the experimental NH₃ sensing parameters shown in Table 5: (a) a plot of resistance ratio versus τ for Da ranging from 10⁻⁴ to 10⁴ (log increment of 0.4); and (b) a plot of resistance ratios versus Da for τ ranging from 0 to 20 (increment of 0.5 between τ=1 and τ=5), in which dotted lines show the contours with constant ρDa values, and the open and filled circles indicate the locations of the 620 nm and 450 nm fibers, respectively, at t=60 s, and the arrows indicate the optimization trajectories for the examples discussed in the text.

### DETAILED DESCRIPTION

### Overview

In certain embodiments, the invention relates to a continuous, submicron diameter fiber, comprising intrinsically conductive polymers (ICPs), as a chemiresistive sensor for gases of industrial or biological relevance. In certain embodiments, the sensor is a p-type semiconductor comprising an ICP and a dopant, whose conductivity is reduced when exposed to an electron-donating vapor species like ammonia. In certain embodiments, the sensor is a n-type semiconductor composed of an ICP without a dopant, whose conductivity is increased when exposed to an electron-withdrawing vapor species like NO₂ (which acts like a dopant for the ICP). In certain embodiments, the fibers are produced by electrospinning so that they have small diameters and large specific surface areas. In certain embodiments, the fiber is used as an individual fiber. In certain embodiments, the fiber is used as a collection of fibers in the form of a bundle or a nonwoven mat. In certain embodiments, the response time of the sensors is very good while operated at room temperature (i.e., about 23° C.), which is attributed to the small diameter (less than 1 micrometer) and high specific surface area of the fibers (estimated >10 m²/g). In certain embodiments, the operating temperature of the device can be elevated to further enhance response speed, with the optimization procedure to be followed apparent to one skilled in the art. In certain embodiments, the sensitivity of the sensors is very good to exceptional (in the case of the n-type sensor), where changes in resistivity spanning several orders of magnitude can be observed. While not wishing to be bound by any particular theory, the high sensitivity is attributed to the nature of the ICP, whose conductivity spans many orders of magnitude based on dopant concentration (e.g., 10⁻¹⁰ to 10³ S/cm for polyaniline (PAni) doped with HCSA).

In certain embodiments, the ICP is an ICP having at least one substituent, such as a poly(3-alkylthiophene) (e.g., poly(3-methylthiophene), poly(3-butylthiophene), poly(3-hexylthiophene), or poly(3-octylthiophene)), poly(3-(octyloxy)-4-methylthiophene), poly(3-(4-octylphenyl)thiophene), or poly(N-(2-cyanoethyl)pyrrole. Other derivatives are also contemplated, including alkyl, alkenyl, alkynyl, halo, haloalkyl, hydroxy, alkoxy, alkenyloxy, alkynyloxy, carbocyclyloxy, heterocyclyloxy, haloalkoxy, sulfhydryl, alkylthio, haloalkylthio, alkenylthio, alkynylthio, sulfonic acid, alkylsulfonyl, haloalkylsulfonyl, alkenylsulfonyl, alkynylsulfonyl, alkoxysulfonyl, haloalkoxysulfonyl, alkenyloxysulfonyl, alkynyloxysulfonyl, aminosulfonyl, sulfinic acid, alkylsulfinyl, haloalkylsulfinyl, alkenylsulfinyl, alkynylsulfinyl, alkoxysulfinyl, haloalkoxysulfinyl, alkenyloxysulfinyl, alkynyloxysulfinyl, aminosulfinyl, formyl, alkylcarbonyl, haloalkylcarbonyl, alkenylcarbonyl, alkynylcarbonyl, carboxy, alkoxycarbonyl, haloalkoxycarbonyl, alkenyloxycarbonyl, alkynyloxycarbonyl, alkylcarbonyloxy, halo alkylcarbonyloxy, alkenylcarbonyloxy, alkynylcarbonyloxy, alkylsulfonyloxy, halo alkylsulfonyloxy, alkenylsulfonyloxy, alkynylsulfonyloxy, halo alkoxysulfonyloxy, alkenyloxysulfonyloxy, alkynyloxysulfonyloxy, alkylsulfinyloxy, halo alkylsulfinyloxy, alkenylsulfinyloxy, alkynylsulfinyloxy, alkoxysulfinyloxy, haloalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, or silyloxy derivatives of ICPs.

In certain embodiments, the invention relates to sensors comprising electrospun PAni fibers. In certain embodiments, the PAni fibers perform effectively as nanoscale sensors for both ammonia (NH₃) and nitrogen dioxide (NO₂) gases. In certain embodiments, the PAni fibers exhibit high sensitivities and fast response times.

In certain embodiments, the fibers of the invention exhibit a response ratio up to almost 60-fold for doped PAni sensing of NH₃ up to 700 ppm. In certain embodiments, the fibers of the invention exhibit a response ratio of more than five orders of magnitude for NO₂ sensing by undoped PAni fibers at concentrations as low as 50 ppm.

In certain embodiments, the characteristic response times for sensing are on the order of 1 to 2 minutes.

In certain embodiments, the invention relates to a method of making any one of the fibers mentioned herein by coaxial electrospinning. In certain embodiments, this technique enables the preparation of submicron fibers of pure ICP (e.g., polyaniline with or without dopant) that does not involve blending with other polymers. In certain embodiments, this is significant because blending generally reduces the range of conductivity accessible to a sensor. In certain embodiments, the fibers are fabricated by coaxial electrospinning, and subsequent removal of the shell by dissolution.

In certain embodiments, the invention relates to a method of making any one of the fibers mentioned herein by centrifugal spinning, melt blowing, electroblowing, or extrusion through specially designed dies.

In certain embodiments, the invention relates to any one of the methods described herein, wherein the fibers are post-processed (e.g., by drawing) to improve their molecular orientation, which increases the range of conductivity (or resistivity) that can be used.

In certain embodiments, the invention relates to a method of detecting a quantity of a gas. In certain embodiments, the method is a method of detecting an industrial gas leak. In certain embodiments, the method is a method of detecting a trace gas in the environment. In certain embodiments, the method is a method of detecting a trace gas in the breath of a subject. For example, hydrogen sulfide has recently been identified as an important signaling molecule in human physiological and pathological processes, such as cell growth regulation, cardiovascular protection, angiogenesis, and Alzheimer's disease. Ammonia (NH₃) is a biomarker for kidney disorder, carbon monoxide is a biomarker for pulmonary disease, and nitrogen monoxide is a biomarker for asthma (see Choi et al, ACS Appl. Mater. Interf. 2014, 6, 2588-2597).

For example, both NO₂ and NH₃ gases are found in a variety of industrial environments. In certain embodiments, oxidizing gases that may be detected by methods of the invention are selected from the group consisting of: HCl, CO₂, O₃, H₂S and SO₂. In certain embodiments, reducing gases that may be detected by methods of the invention are selected from the group consisting of: H₂, NO, and CO.

In certain embodiments, the method is a method of optimizing an automotive emissions control system. Automotive emissions control systems, including diesel and lean burn engines, use Selective Catalytic Reduction (SCR) to remove NOx by reaction with ammonia, e.g.,

2NO₂+4NH₃+O₂→3N₂+6H₂O

For such systems, sensors detect both ammonia and NOx in the exhaust as a means of optimally controlling the amount of ammonia injected into the system and insuring a minimal amount of NOx and NH₃ breaking through the system and being emitted into the atmosphere. In certain embodiments, the invention relates to a sensor for use in such systems.

In certain embodiments, a reaction-diffusion model is used to characterize the reaction kinetics and molecular diffusivities of the gases within the fibers, and to design future materials for optimal sensing performance under various conditions of fiber size, gas concentration, reaction kinetics, and gas adsorption into fibers.

### Exemplary Methods of Sensing

In certain embodiments, the invention relates to a method of detecting a gas in a sample, comprising the steps of:

optionally determining the electrical resistance (R₀) or electrical conductance of a fiber;

contacting with the fiber a quantity of the sample; and

after a period of time, determining the electrical resistance (Rₑₓ) or electrical conductance of the fiber.

In certain embodiments, the invention relates to a method of detecting and quantifying a gas in a sample, comprising the steps of:

(a) optionally determining the electrical resistance (R₀) or electrical conductance of a fiber;

(b) contacting with the fiber a first standard sample, wherein the concentration of the gas in the first standard sample is known;

(c) after a period of time, determining the electrical resistance or electrical conductance of the fiber;

(d) contacting with the fiber a second standard sample, wherein the concentration of the gas in the second standard sample is known; and the concentration of the gas in the second standard sample is different from the concentration of the gas in the first standard sample;

(e) after a period of time, determining the electrical resistance or electrical conductance of the fiber;

(f) contacting with the fiber a quantity of the sample; and

(g) after a period of time, determining the electrical resistance (Rₑₓ) or electrical conductance of the fiber.

In certain embodiments, the invention relates to any one of the aforementioned methods, wherein the gas is an oxidizing gas selected from the group consisting of: NO₂, HCl, CO₂, O₃, H₂S, and SO₂.

In certain embodiments, the invention relates to any one of the aforementioned methods, wherein the gas is a reducing gas selected from the group consisting of: NH₃, H₂, NO, and CO.

In certain embodiments, the invention relates to any one of the aforementioned methods, wherein the fiber comprises a polymer selected from the group consisting of: a polyaniline, a polyacetylene, a polypyrrole, a polythiophene, and a poly(3,4-ethylenedioxythiophene) (PEDOT).

In certain embodiments, the invention relates to any one of the aforementioned methods, wherein the fiber consists essentially of a polymer selected from the group consisting of: a polyaniline, a polyacetylene, a polypyrrole, a polythiophene, and a poly(3,4-ethylenedioxythiophene) (PEDOT).

In certain embodiments, the invention relates to any one of the aforementioned methods, wherein the fiber consists of a polymer selected from the group consisting of: a polyaniline, a polyacetylene, a polypyrrole, a polythiophene, and a poly(3,4-ethylenedioxythiophene) (PEDOT).

In certain embodiments, the invention relates to any one of the aforementioned methods, wherein the fiber consists essentially of a polymer selected from the group consisting of: a polyaniline, a polyacetylene, a polypyrrole, a polythiophene, and a poly(3,4-ethylenedioxythiophene) (PEDOT); and the gas is an oxidizing gas selected from the group consisting of: NO₂, HCl, CO₂, O₃, H₂S, and SO₂. Oxidizing gases are electron-withdrawing and thus acts as a dopant to increase the charge carrier concentration of the polymer. Consequently, upon exposure to an oxidizing gas, the measured resistance of an undoped polymer sample decreases. In certain embodiments, changes in resistance are reported as −ΔR/Rₑₓ, in units of ppm⁻¹, where ΔR=Rₑₓ−R₀, R₀ is the measured initial resistance prior to any exposure to the gas, and Rₑₓ is the measured resistance upon exposure.

In certain embodiments, the invention relates to any one of the aforementioned methods, wherein the fiber comprises a dopant and a polymer; and the polymer is selected from the group consisting of: a polyaniline, a polyacetylene, a polypyrrole, a polythiophene, and a poly(3,4-ethylenedioxythiophene) (PEDOT).

In certain embodiments, the invention relates to any one of the aforementioned methods, wherein the fiber consists essentially of a dopant and a polymer; and the polymer is selected from the group consisting of: a polyaniline, a polyacetylene, a polypyrrole, a polythiophene, and a poly(3,4-ethylenedioxythiophene) (PEDOT).

In certain embodiments, the invention relates to any one of the aforementioned methods, wherein the fiber consists of a dopant and a polymer; and the polymer is selected from the group consisting of: a polyaniline, a polyacetylene, a polypyrrole, a polythiophene, and a poly(3,4-ethylenedioxythiophene) (PEDOT).

In certain embodiments, the invention relates to any one of the aforementioned methods, wherein the fiber consists essentially of a dopant and a polymer; and the polymer is selected from the group consisting of: a polyaniline, a polyacetylene, a polypyrrole, a polythiophene, and a poly(3,4-ethylenedioxythiophene) (PEDOT); and the gas is a reducing gas selected from the group consisting of: NH₃, H₂ and CO. These polymers, when doped, exhibit p-type semiconductor characteristics, so exposure to electron-donating species, such as NH₃, gives rise to a decrease in the charge-carrier concentrations and thus an increase in the measured resistance. In certain embodiments, changes in resistance are reported as ΔR/R₀, in units of ppm⁻¹, where ΔR=Rₑₓ−R₀, R₀ is the measured initial resistance prior to any exposure to the gas, and Rₑₓ is the measured resistance upon exposure.

In certain embodiments, the invention relates to any one of the aforementioned methods, wherein the fiber has a long length.

In certain embodiments, the invention relates to any one of the aforementioned methods, wherein the fiber has at least one dimension, e.g., a width or diameter, of about 1 nm to about 10 μm. In certain embodiments, the fibers are ultra-fine and can provide a high weight loading when taken collectively. In certain embodiments, the diameter of the fiber is about 200 nm to about 1200 nm. In certain embodiments, the diameter of the fiber is about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, about 1000 nm, about 1025 nm, about 1050 nm, about 1075 nm, about 1100 nm, about 1125 nm, about 1150 nm, about 1175 nm, or about 1200 nm.

In certain embodiments, the invention relates to any one of the aforementioned methods, wherein the dopant is selected form the group consisting of HCSA, HCl, HClO₄, HI, FeCl₃, 4-dodecylbenzenesulfonic acid, p-toluenesulfonic acid, and dinonylnaphthalenedisulfonic acid.

### Exemplary Methods of Fabrication

In certain embodiments, the invention relates to a method of forming a plurality of core-shell electrospun fibers.

In certain embodiments, the invention relates to a method of forming a plurality of core-shell electrospun fibers, comprising the steps of:

contacting an electrode with a first fluid and a second fluid;

positioning the electrode at a distance from a grounded collection surface; and

applying an electric voltage to the electrode, thereby forming an electrified jet at the surface of the electrode;

wherein the electrified jet comprises a core layer and a shell layer; and the plurality of core-shell fibers is deposited on the grounded collection surface.

In certain embodiments, the invention relates to any one of the aforementioned methods, further comprising the step of: removing the shell from the core-shell fiber, thereby producing a polymeric fiber. In certain embodiments, the invention relates to any one of the aforementioned methods, wherein the shell is removed from the core-shell fiber by selective dissolution. In certain embodiments, the invention relates to any one of the aforementioned methods, wherein the shell is removed from the core-shell fiber by selective dissolution in a third solvent. In certain embodiments, the invention relates to any one of the aforementioned methods, wherein the third solvent is isopropyl alcohol.

In certain embodiments, the invention relates to any one of the aforementioned methods, wherein the first fluid comprises a first solvent and a polymer selected from the group consisting of: a polyacetylene, a polypyrrole, a polythiophene, and a poly(3,4-ethylenedioxythiophene) (PEDOT).

In certain embodiments, the invention relates to any one of the aforementioned methods, wherein the first fluid comprises a first solvent, a dopant, and a polymer selected from the group consisting of: a polyacetylene, a polypyrrole, a polythiophene, and a poly(3,4-ethylenedioxythiophene) (PEDOT).

In certain embodiments, the invention relates to any one of the aforementioned methods, wherein the first fluid consists essentially of a first solvent and a polymer selected from the group consisting of: a polyacetylene, a polypyrrole, a polythiophene, and a poly(3,4-ethylenedioxythiophene) (PEDOT).

In certain embodiments, the invention relates to any one of the aforementioned methods, wherein the first fluid consists essentially of a first solvent, a dopant, and a polymer selected from the group consisting of: a polyacetylene, a polypyrrole, a polythiophene, and a poly(3,4-ethylenedioxythiophene) (PEDOT).

In certain embodiments, the invention relates to any one of the aforementioned methods, wherein the first fluid consists of a first solvent and a polymer selected from the group consisting of: a polyacetylene, a polypyrrole, a polythiophene, and a poly(3,4-ethylenedioxythiophene) (PEDOT).

In certain embodiments, the invention relates to any one of the aforementioned methods, wherein the first fluid consists of a first solvent, a dopant, and a polymer selected from the group consisting of: a polyacetylene, a polypyrrole, a polythiophene, and a poly(3,4-ethylenedioxythiophene) (PEDOT).

In certain embodiments, the invention relates to any one of the aforementioned methods, wherein the first solvent is chloroform, dimethylformamide (DMF), or a combination thereof.

In certain embodiments, the invention relates to any one of the aforementioned methods, wherein the polymer is present in the first fluid in about 0.5, about 1.0, about 1.5, about 2.0, about 2.5, about 3.0, about 3.5, about 4.0, about 4.5, or about 5.0 wt %.

In certain embodiments, the invention relates to any one of the aforementioned methods, wherein the molar ratio of dopant-to-polymer is about 0, about 0.25, about 0.5, about 0.75, or about 1.

In certain embodiments, the invention relates to any one of the aforementioned methods, wherein the molecular weight of the polymer is from about 10,000 Da to about 100,000 Da.

In certain embodiments, the invention relates to any one of the aforementioned methods, wherein the second fluid comprises a second polymer and a second solvent. In certain embodiments, the invention relates to any one of the aforementioned methods, wherein the second polymer is polymethylmethacrylate.

In certain embodiments, the invention relates to any one of the aforementioned methods, wherein the molecular weight of the polymethylmethacrylate is from about 100,000 Da to about 1,000,000 Da.

In certain embodiments, the invention relates to any one of the aforementioned methods, wherein the second solvent is DMF.

In certain embodiments, the invention relates to any one of the aforementioned methods, wherein the second polymer is present in the second fluid in 10, about 11, about 12, about 13, about 14, about 15, about 16, about 17, about 18, about 19, or about 20 wt %.

In certain embodiments, the invention relates to any one of the aforementioned methods, wherein the electrified jets cool to form core-shell fibers.

In certain embodiments, the invention relates to any one of the aforementioned methods, wherein a solvent in the electrified jets evaporates, thereby forming the core-shell fibers.

In certain embodiments, the invention relates to any one of the aforementioned methods, wherein the electric voltage is about 1 kV to about 100 kV. In certain embodiments, the invention relates to any one of the aforementioned methods, wherein the electric voltage is about 13 kV, about 14 kV, about 15 kV, about 16 kV, about 17 kV, about 18 kV, about 19 kV, about 20 kV, about 21 kV, about 22 kV, about 23 kV, about 24 kV, about 25 kV, about 26 kV, about 27 kV, about 28 kV, about 29 kV, about 30 kV, about 31 kV, about 32 kV, about 33 kV, about 34 kV, about 35 kV, about 36 kV, about 37 kV, about 38 kV, about 39 kV, about 40 kV, about 41 kV, about 42 kV, about 43 kV, about 44 kV, about 45 kV, about 46 kV, about 47 kV, about 48 kV, about 49 kV, about 50 kV, about 51 kV, about 52 kV, about 53 kV, about 54 kV, about 55 kV, about 56 kV, about 57 kV, or about 58 kV.

In certain embodiments, the invention relates to any one of the aforementioned methods, wherein the grounded collection surface is a grounded collection plate, a grounded rotating drum, a grounded rotating wheel, or a grounded conveyor belt.

In certain embodiments, the invention relates to any one of the aforementioned methods, wherein the distance between the electrode and the grounded collection surface is about 1 to about 100 centimeters. In certain embodiments, the invention relates to any one of the aforementioned methods, wherein the distance between the electrode and the grounded collection surface is about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, or about 40 cm.

In certain embodiments, the invention relates to any one of the aforementioned methods, wherein the grounded collection surface comprises various geometries (e.g., rectangular, circular, triangular, etc.), rotating drum/rod, wire mesh, air gaps, or other 3-D collectors including spheres, pyramids, etc.

In certain embodiments, the invention relates to any one of the aforementioned methods, wherein the method is described in U.S. Patent Application Publication No. 2012/0208421, which is hereby incorporated by reference in its entirety.

In certain embodiments, the invention relates to any one of the aforementioned methods, wherein the method is described in PCT Application Serial No. PCT/US13/068667, which is hereby incorporated by reference in its entirety.

### Exemplary Fibers

In certain embodiments, the invention relates to a fiber comprising an intrinsically conducting polymer.

In certain embodiments, the invention relates to a fiber comprising an intrinsically conducting polymer and a dopant.

In certain embodiments, the invention relates to a fiber comprising a polymer selected from the group consisting of: a polyacetylene, a polypyrrole, a polythiophene, and a poly(3,4-ethylenedioxythiophene) (PEDOT).

In certain embodiments, the invention relates to a fiber comprising a dopant and a polymer, wherein the polymer is selected from the group consisting of: a polyacetylene, a polypyrrole, a polythiophene, and a poly(3,4-ethylenedioxythiophene) (PEDOT).

In certain embodiments, the invention relates to a fiber consisting essentially of an intrinsically conducting polymer.

In certain embodiments, the invention relates to a fiber consisting essentially of an intrinsically conducting polymer and a dopant.

In certain embodiments, the invention relates to a fiber consisting essentially of a polymer selected from the group consisting of: a polyacetylene, a polypyrrole, a polythiophene, and a poly(3,4-ethylenedioxythiophene) (PEDOT).

In certain embodiments, the invention relates to a fiber consisting essentially of a dopant and a polymer, wherein the polymer is selected from the group consisting of: a polyacetylene, a polypyrrole, a polythiophene, and a poly(3,4-ethylenedioxythiophene) (PEDOT).

In certain embodiments, the invention relates to a fiber consisting of an intrinsically conducting polymer.

In certain embodiments, the invention relates to a fiber consisting of an intrinsically conducting polymer and a dopant.

In certain embodiments, the invention relates to a fiber consisting of a polymer selected from the group consisting of: a polyacetylene, a polypyrrole, a polythiophene, and a poly(3,4-ethylenedioxythiophene) (PEDOT).

In certain embodiments, the invention relates to a fiber consisting of a dopant and a polymer, wherein the polymer is selected from the group consisting of: a polyacetylene, a polypyrrole, a polythiophene, and a poly(3,4-ethylenedioxythiophene) (PEDOT).

In certain embodiments, the invention relates to any one of the aforementioned fibers, wherein the fiber has a long length.

In certain embodiments, the invention relates to any one of the aforementioned fibers, wherein the fiber has at least one dimension, e.g., a width or diameter, of about 1 nm to about 10 μm. In certain embodiments, the fibers are ultra-fine and can provide a high weight loading when taken collectively. In certain embodiments, the diameter of the fiber is about 200 nm to about 1200 nm. In certain embodiments, the diameter of the fiber is about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, about 1000 nm, about 1025 nm, about 1050 nm, about 1075 nm, about 1100 nm, about 1125 nm, about 1150 nm, about 1175 nm, or about 1200 nm.

In certain embodiments, the invention relates to any one of the aforementioned fibers, wherein the dopant is selected form the group consisting of HCSA, HCl, HClO₄, HI, FeCl₃, 4-dodecylbenzenesulfonic acid, p-toluenesulfonic acid, and dinonylnaphthalenedisulfonic acid.

In certain embodiments, the invention relates to any one of the aforementioned fibers, wherein the intrinsically conducting polymer is not polyaniline.

### Exemplary Sensors

In certain embodiments, the invention relates to a sensor comprising any one of the aforementioned fibers.

In certain embodiments, the invention relates to a sensor comprising a plurality of any one of the aforementioned fibers configured as a non-woven material. In certain embodiments, the non-woven material has uniform, well-controlled surface morphology. In certain embodiments, non-woven materials has tunable properties including, but not limited to, mechanical robustness, surface properties, and/or electrical-, thermal-, and/or chemical properties.

In certain embodiments, the non-woven material has desirable surface energy.

In certain embodiments, the non-woven material has desirable mechanical properties (e.g., tensile strength, elongation %, toughness, or initial modulus).

In certain embodiments, the non-woven material has desirable thermal diffusivity.

### EXEMPLIFICATION

The invention now being generally described, it will be more readily understood by reference to the following examples, which are included merely for purposes of illustration of certain aspects and embodiments of the invention, and are not intended to limit the invention.

### Example 1

### General Material and Methods

Material

Polyaniline (PAni, emeraldine base, M_{w}=65,000) was purchased from Sigma-Aldrich, Inc. The dopant, (+)-camphor-10-sulfonic acid (HCSA), was obtained from Fluka Analytical Chemicals. Poly(methyl methacrylate) (PMMA, M_{w}=960,000 g/mol) was purchased from Scientific Polymer Products Inc. The N,N-dimethylformamide (DMF) and isopropyl alcohol (IPA) used were OmniSolv® solvents from EMD Chemicals. Chloroform was purchased from Mallinckrodt Chemical Inc. Certified pre-mixed gases (1000 ppm NH₃ in dry nitrogen, 100 ppm NO₂ in dry air, dry nitrogen and dry air) were all purchased from Airgas, Inc. All materials were used without further purification.

Sample Preparation: Electrospinning

Core-shell fibers were prepared by the method of coaxial electrospinning, followed by selective removal of the shell. The core fluid was 2 wt % PAni, blended with various amounts of HCSA, dissolved in a 5:1 mixture by weight of chloroform and DMF; the shell fluid was 15 wt % PMMA in DMF. The core and shell fluid flow rates were 0.010 mL/min and 0.050 mL/min, controlled independently by two syringe pumps. The applied voltage was 34 kV, and the distance between the spinneret and collection plate was 30 cm. After the fibers were formed, the resultant fibers and mats were then immersed in IPA for one hour with gentle stirring, so that the PMMA shell component was removed, leaving intact the doped PAni fiber cores. X-ray photoelectron spectroscopy (XPS) and differential scanning calorimetry (DSC) were used to confirm the removal of the PMMA shell. The core-shell fibers were also post-processed to stretch the fibers longitudinally, where it was shown that the molecular orientation of the PAni molecules increased with increasing longitudinal strain in the resultant fibers.

Fiber Electrical Conductivity

Fibers were electrospun onto interdigitated Pt electrodes (IDE, ABTech) with 50 sets of interdigitated fingers, and finger width and spacing ranging from 5 to 20 μm. Their electrical properties were measured with an impedance analyzer (Solartron 1260/1287A), with resistance values extracted from the frequency-dependent Nyquist plots. The measurements were performed in controlled environments at room temperature and 20% relative humidity. The fiber electrical conductivity (σ_{f}), with correction for contact resistance R_{f0}, was calculated according to Equation 1

$${σ}_{f} = \frac{4 δ}{π {d}^{2} ( {R}_{f} - {R}_{f 0} )} \qquad ( 1 )$$

where the single-fiber resistance, R_{f}=(RN), R is the resistance measured on the IDE, N is the number of parallel pathways formed by fibers on the IDE bridging over the interdigitated fingers as observed by optical microscopy, d is the average fiber diameter obtained by scanning electron microscopy (SEM) on the as-spun fibers, and δ is the finger spacing (inter-electrode distance) of the IDE.

Gas Sensing

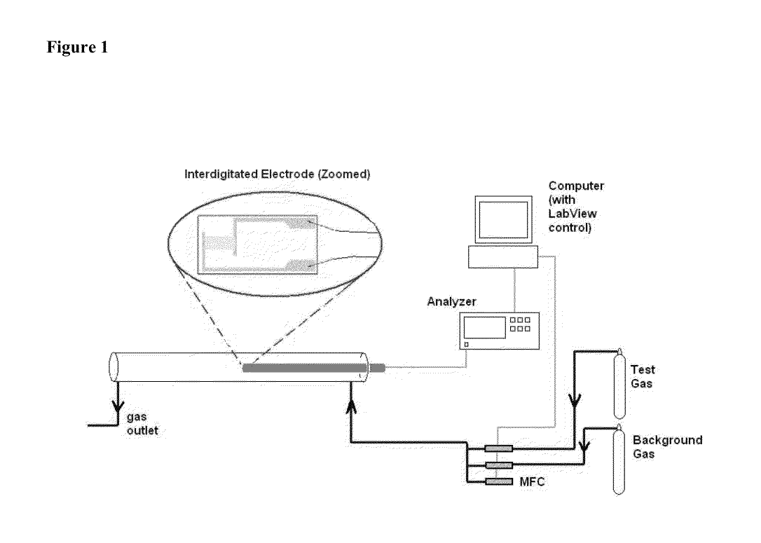

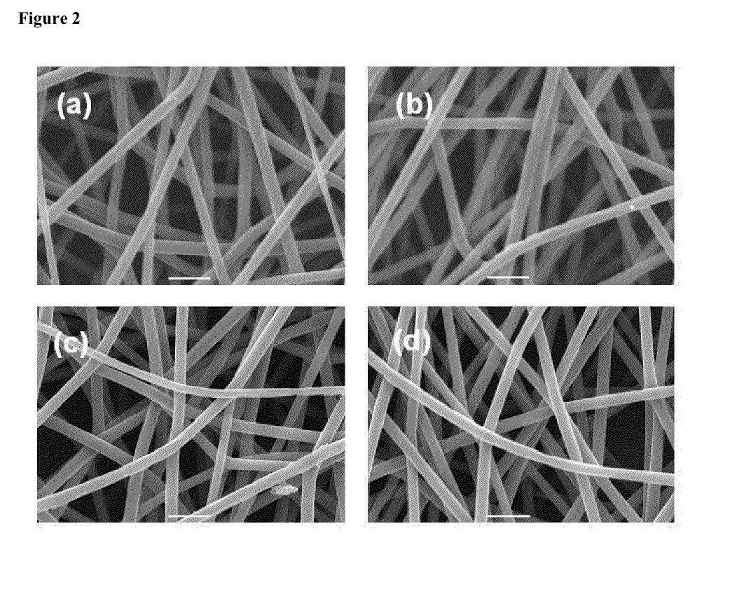

Gas sensing tests were conducted in a quartz tube placed inside a Lindberg Blue TF 55035A furnace, and exposure of the samples to different gases was achieved using mass flow controllers (MKS Instruments) on separate streams of test gases and inert background gases. The temperature of the tube furnace remained at room temperature (20° C.) and was not adjusted. The experiments were conducted inside the furnace to avoid any spurious changes in charge carrier concentrations due to external illumination. The setup is illustrated in FIG. 1.

All experiments were conducted with a constant total gas flow rate of 200 sccm. For NH₃ sensing, a certified premixed gas containing 1000 ppm of NH₃ in dry nitrogen was diluted with additional dry nitrogen gas by MFC's to a concentration in the range of 10 to 700 ppm of NH₃; for NO₂ sensing, a certified premixed gas containing 100 ppm of NO₂ in dry air was diluted with additional dry air to a concentration in the range of 1 to 50 ppm of NO₂. For each sample, about 10 aligned electrospun fibers were deposited on an IDE with 10 μm finger spacings. The contacts from the interdigitated electrodes to the testing apparatus were made by platinum wires. The DC resistances between the measurement portals in the quartz tube were measured by an Agilent HP34970A data acquisition system controlled by a LabView program and interface.

PAni doped with HCSA exhibits p-type semiconductor characteristics, so exposure to electron-donating species such as NH₃ gives rise to a decrease in the charge-carrier concentrations and thus an increase in the measured resistance. For NH₃ sensing, changes in resistance of doped PAni fibers are therefore reported as ΔR/R₀, where ΔR=Rₑₓ−R₀, R₀ is the measured initial resistance prior to any exposure to NH₃, and Rₑₓ is the measured resistance upon exposure. In contrast to NH₃, NO₂ is electron-withdrawing and thus acts as dopant in lieu of HCSA to increase the charge carrier concentration of p-type PAni. Consequently, upon exposure to NO₂ the measured resistance of an undoped PAni sample decreases. For NO₂ sensing, changes in resistance are therefore reported as −ΔR/Rₑₓ. The sensitivity of the materials for gas sensing, in units of ppm⁻¹, is defined as the ratio between ΔR/R₀ (for ammonia) or −ΔR/Rₑₓ (for nitrogen dioxide) and the concentration of the test gas in ppm, in the range of low test gas concentration where there is a linear relation between these two quantities.

QCM Analysis

A quartz crystal microbalance with dissipation monitoring (QCM-D, Q-sense) was used to measure the change in mass of electrospun fibers due to absorption of NH₃ during exposure to a gas of fixed NH₃ concentration. A thin layer of as-electrospun PAni fibers (about 10 μm in thickness) was deposited on the quartz crystal resonator with gold electrodes. The coated resonator was placed in the Q-sense flow cell with a blank crystal as a reference. A mixture of NH₃ and nitrogen gas was introduced to the flow cell through a flow controller. Changes in both frequency and dissipation for the crystal harmonics were then recorded, and equilibrium values were obtained after 10 minutes of equilibration. The change in mass was calculated using the Saurbrey relationship

$$Δ m = - C \frac{1}{n} Δ f \qquad ( 2 )$$

where Δm is the change in mass, C is a constant dependent on the crystal and C=17.7 ng s cm⁻² in this case, n is the harmonic overtone, and Δf is the frequency change. For calculations, the third, fifth, and seventh harmonics (n=3, 5, and 7) were used to obtain an averaged value for the changes in mass.

### Example 2

### Morphologies and Electrical Properties of as-Electrospun Fibers

FIG. 2 shows representative images of the PAni/HCSA fibers after coaxial electrospinning and removal of the PMMA shell component by dissolution in IPA. The fibers are confirmed to be smooth, relatively uniform in diameter and continuous. No significant difference in fiber diameters is observed for fibers prepared with molar ratios of HCSA to PAni of 0, 0.25, 0.50, 0.75 or 1. However, the electrical conductivities of the fibers increase exponentially with increasing molar ratio of HCSA to PAni, as shown in Table 1. This trend is consistent with previous observations for PAni pellets with different doping levels.

TABLE 1
Diameter and Conductivity of As-spun
PAni/HCSA Fibers after Removing Shell

| | | Electrical |
|---|---|---|
| [HCSA]/[PAni] | | Conductivity, σ_{f} |
| Mole Ratio | Diameter, d (nm) | (S/cm) |
| 0 | 650 ± 110 | (2.0 ± 0.6) × 10⁻⁶ |
| 0.25 | 670 ± 120 | 0.0022 ± 0.0008 |
| 0.50 | 600 ± 90 | 0.18 ± 0.05 |
| 0.75 | 650 ± 110 | 2.3 ± 0.9 |
| 1.0 | 620 ± 160 | 50 ± 30 |

### Example 3

### Ammonia Sensing

The PAni fibers with HCSA:PAni mole ratio of 1 were used for sensing experiments with NH₃ for concentrations from 10 to 700 ppm. Both as-electrospun fibers (average fiber diameter=620 nm) and after solid-state drawing (average fiber diameter=450 nm) were tested.

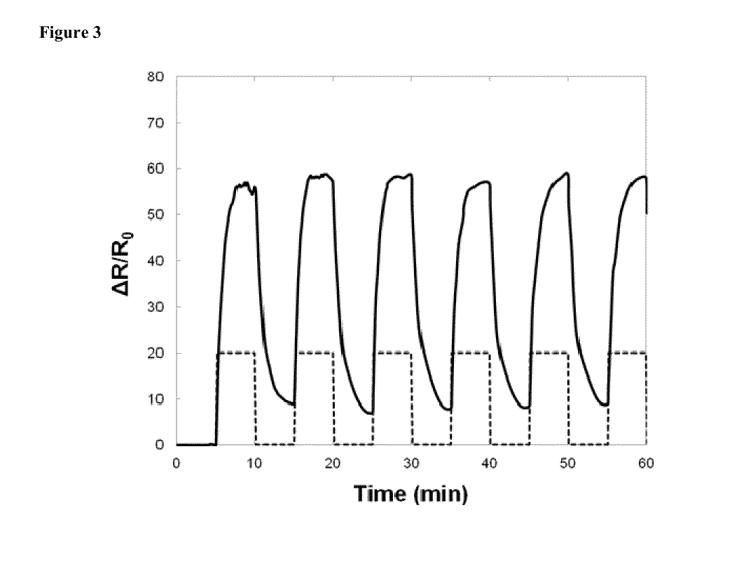

The gas sensing responses are fast, as demonstrated by a representative plot of ΔR/R₀ versus time for drawn PAni fibers with diameter of 450 nm shown in FIG. 3. The PAni fibers were exposed to repeated cycles of 5 min exposure to a gas stream of 500 ppm of ammonia (balance nitrogen) followed by 5 min of nitrogen purging. The response time is defined as the time required for the signal to reach 1/e of its steady state value. For the case shown in FIG. 3, the average response time is 45±3 seconds upon exposure, and 63±9 seconds for recovery upon purging.

The results also show that the measurement was reasonably reversible; the maximum ΔR/R₀ value did not vary much over multiple cycles of exposure to the same concentration of gases, so that the fibers can be used multiple times for sensing. However, there was an increase of baseline resistance after the first cycle in some cases, as seen in the case shown in FIG. 3, indicating that nitrogen purging alone is not enough to return the fibers to the original state, i.e., some ammonia molecules have irreversibly reacted with or bound to the fibers. The baseline does not increase after subsequent cycles, so that the sensing measurements are reversible after the first cycle of exposure in all cases.

TABLE 2
Characteristic Response Times of As-Spun and
Solid-State-Drawn PAni/HCSA Fibers with Mole Ratio
[HCSA]/[PAni] = 1.0 during Ammonia Exposure and Nitrogen Purge

| NH₃ | Response time (s) for | | Response time (s) for | |
|---|---|---|---|---|
| Concentration | as-spun PAni fiber | | solid-state drawn PAni fiber | |
| (ppm) | Exposure | Purging | Exposure | Purging |
| 20 | 84 ± 6 | 133 ± 8 | 82 ± 3 | 109 ± 9 |
| 50 | 82 ± 4 | 92 ± 4 | 67 ± 5 | 84 ± 4 |
| 100 | 66 ± 6 | 75 ± 2 | 59 ± 8 | 83 ± 5 |
| 500 | 43 ± 3 | 61 ± 8 | 45 ± 3 | 63 ± 9 |
| 700 | 31 ± 4 | 53 ± 6 | 28 ± 5 | 47 ± 7 |

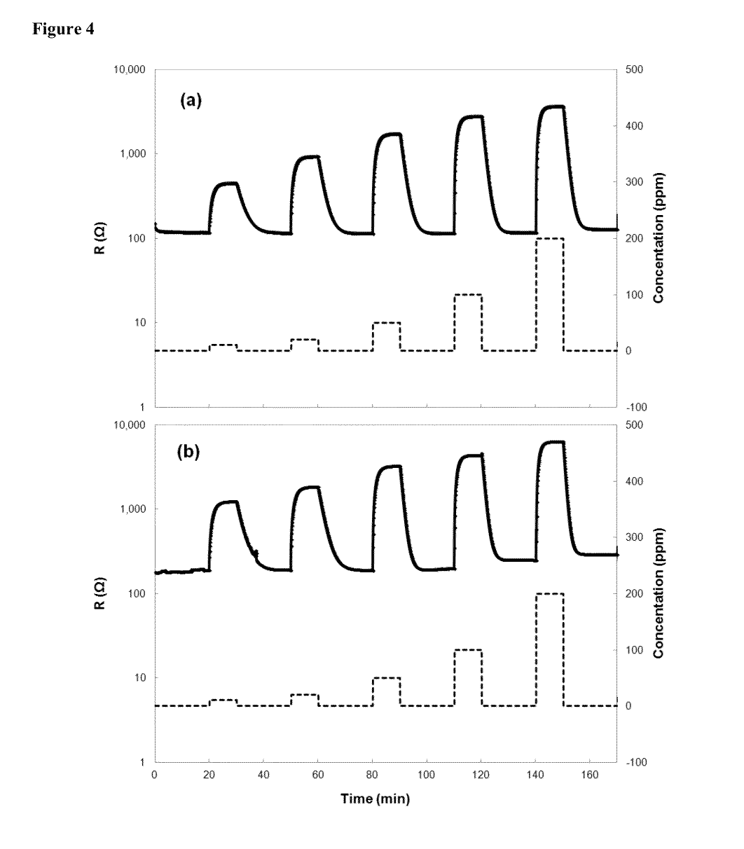

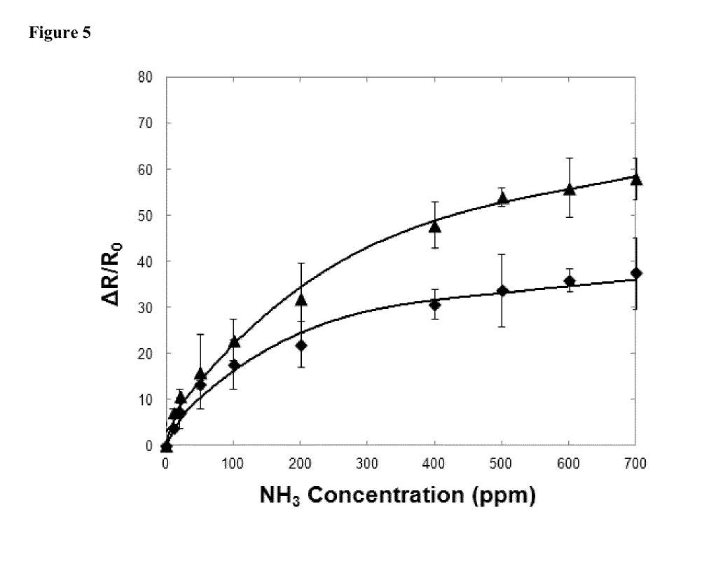

PAni fibers were then subjected to longer exposures of ammonia for concentrations ranging from 10 to 700 ppm. Table 2 lists the characteristic response times (averaged over at least three cycles) of the as-spun and solid-state drawn PAni fibers with different levels of ammonia exposure. Response times decrease monotonically with increasing ammonia concentration. The recovery times with nitrogen purging are significantly longer than the exposure responses times. When comparing the as-spun and solid-state drawn PAni fibers, the drawn fibers tend to have slightly faster response times, but the difference is not significant. The difference could be due to both the smaller fiber diameter and the high level of molecular orientation that comes with solid state drawing. In general, 10 minutes is sufficient for the signals to reach steady state during both exposure and purging, as shown in the time response of fibers to exposure of different concentration of ammonia in FIG. 4.

The steady-state responses after 10 min exposures of ammonia are shown in FIG. 5 as a function of increasing ammonia concentration from 10 to 700 ppm. The measured resistances of both as-spun and solid-state drawn HCSA-doped PAni fibers increase dramatically upon exposure to NH₃. Responses as large as ΔR/R₀=38±8 were observed at 700 ppm ammonia for the as-spun PAni fibers (d=620 nm), and 58±5 for the solid-state drawn PAni fibers (d=450 nm). Such large responses are among the highest thus far reported for PAni or PAni-composite fibers, and are advantageous for gas sensing where signal-to-noise ratios can be an issue. In the linear region of exposure to concentrations below 20 ppm of ammonia, the sensitivity of the 620 nm fiber is 3.5 ppm⁻¹, and the sensitivity of the 450 nm fiber is 5.5 ppm⁻¹, both of which are much higher than the sensitivity of a cast film of the same material with 10 μm thickness, measured at 0.02 ppm⁻¹. The ammonia exposure limit in the United States is 25 ppm over an eight-hour period, and 35 ppm over a short-term exposure. The level of sensitivity exhibited by these fibers is sufficient for rigorous environmental monitoring at these levels.

### Example 4

### Nitrogen Dioxide Sensing

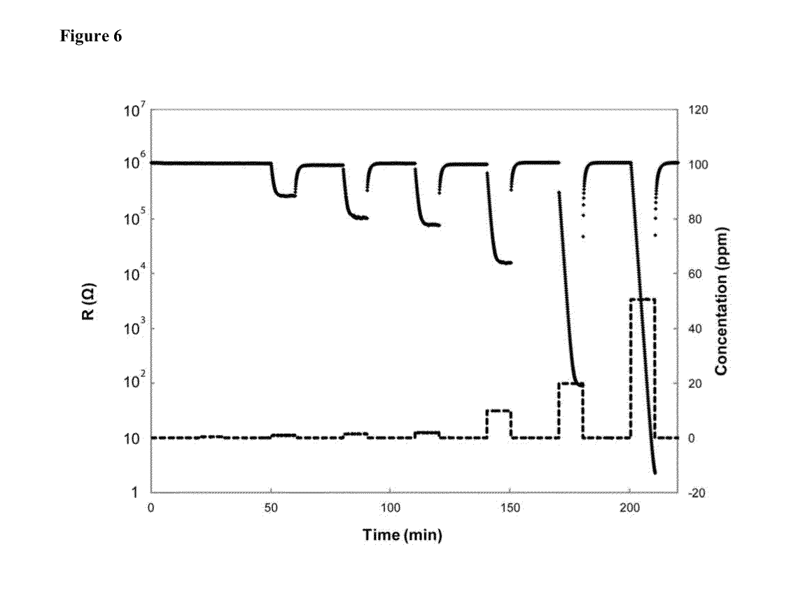

For NO₂ gas sensing, as-spun undoped PAni fibers (i.e., mole ratio [HCSA]/[PAni]=0, d=650 nm) were tested. The representative time response is shown in FIG. 6 for the exposure of undoped PAni electrospun fibers to concentrations of NO₂ between 1 and 50 ppm. Similar to the NH₃ sensing system, the undoped PAni fiber sensor also shows quick response times and good recovery. Table 3 lists the characteristic response times of the undoped PAni fibers to NO₂ exposure. The response times are on the order of 50 s for exposure and 70 s for purging, and do not vary much within the range of concentrations from 1 to 50 ppm.

TABLE 3
Characteristic Response Times of Undoped PAni Fibers during NO₂
Exposure and Purge

| | |
|---|---|
| NO₂ Concentration | Response Time (s) |

| (ppm) | Exposure | Purging |
|---|---|---|
| 1 | 55 ± 5 | 68 ± 8 |
| 2 | 50 ± 9 | 71 ± 6 |
| 5 | 48 ± 3 | 62 ± 5 |
| 10 | 45 ± 3 | 61 ± 8 |
| 20 | 43 ± 3 | 67 ± 8 |
| 50 | 46 ± 5 | 82 ± 9 |

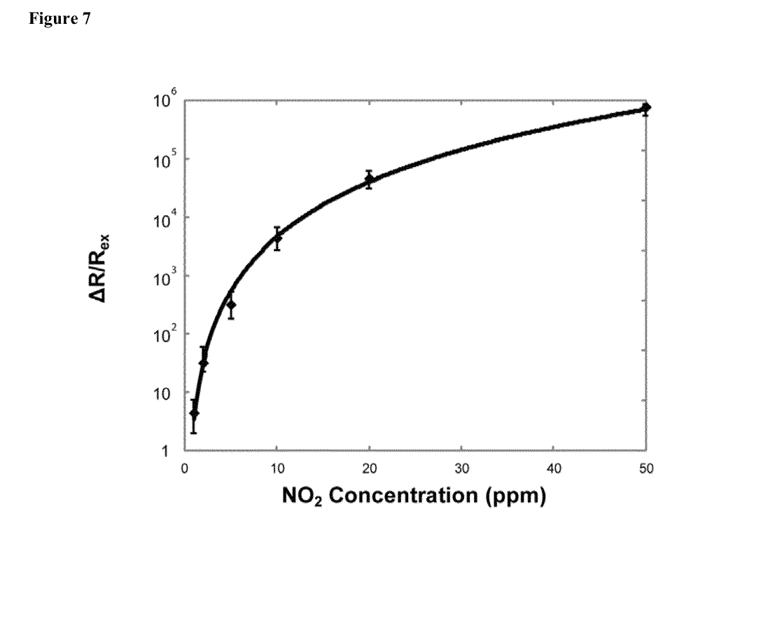

FIG. 7 shows the response of the undoped PAni fibers to NO₂ exposure with concentrations in the range between 1 and 50 ppm. The reported ΔR/Rₑₓ values are taken after 10 minutes of sustained exposure. The resistance decreases remarkably upon exposure to NO₂ concentrations between 1 and 50 ppm. The huge response, up to almost 6 orders of magnitude, indicates that pure PAni fibers can be very effective NO₂ sensors, changing PAni from its undoped, insulating state to almost the fully doped, high conductivity state. The response at 1 ppm is a more than 80% decrease in resistance, indicating very good sensitivity even under exposure to very low concentrations of NO₂. The exposure limit set by the environmental agencies in the US for NO₂ is 50 ppb, which is a concentration too low to be tested directly with the gas composition and flow controllers available for this work. However, based on extrapolations at low NO₂ concentrations, it is reasonable to expect at least a 15% decrease in resistance for these PAni fibers when exposed to 50 ppb NO₂, a response that should be easily detectable. With their large response magnitude and short response time, these PAni fibers can serve as the basis of a very effective nanoscale sensor for NO₂.

### Example 5

### Reaction Diffusion Model

Reaction Equilibrium

A major difference between the experimental results for NH₃ and NO₂ sensing is that ΔR/Rₑₓ for NO₂ undergoes changes in resistance of up to 6 orders of magnitude, while ΔR/R₀ for NH₃ exhibits less than two orders of magnitude change. It is apparent from the values for conductivity in Table 1 that the whole range of doping levels is not being explored in the NH₃ case. The most likely explanation is that ammonia, being a relatively weak base, does not fully deprotonate the doped PAni in the presence of the acidic HCSA, even at concentrations as high as 700 ppm. This can be explained by a reaction equilibrium between the doped PAni and NH₃:

PAni−H⁺+NH₃ [symbol] PAni+NH₄⁺, Scheme 1:

wherein the equilibrium lies somewhere in the middle rather than to either extreme, i.e.,

$$\frac{[ PAni ]}{[ PAni - {H}^{+} ]} = \frac{K [ {NH}_{3} ]}{[ {NH}_{4}^{+} ]} •1 \qquad ( 3 )$$

On the other hand, because the PAni fibers used for nitrogen dioxide sensing were undoped, the incoming NO₂ serves as the only available acidic dopant for PAni; there is no competing strong acid/base in the system. The huge change of conductivity suggests that the reaction is mostly irreversible, or the equilibrium lies very much to the right (K approaches ∞).

Response as a Function of Radial Electrical Conductivities

To characterize the changes in resistance observed in this work, we model the fibers as simple cylindrical elements in which gases diffuse radially into the fiber upon exposure. The model is simplified by assuming that the chemical composition (and thus conductivity) of the fibers varies only with radial position, so that the overall observed change in resistance can be expressed as

$$\frac{{R}_{ex}}{{R}_{0}} = \frac{{σ}_{0} {L}^{2}}{2 {\int }_{0}^{L} σ ( Φ ( r ) ) r d r} \qquad ( 4 )$$

where r is the radial position in the fiber, L is the characteristic length, which is the fiber radius in this case, σ₀ is the fiber conductivity prior to exposure, and σ(Φ(r)) is the radially varying conductivity as a function of concentration of the reactive component in the fiber (Φ(r)) and thus a function of r. This model can be thought of as concentric shells in the fiber forming parallel conducting pathways throughout the length of the fiber, with the inverse of total resistance for the fiber being the sum of the inverse resistances (conductivities) for each concentric shell weighted by its respective cross-sectional area.

Time-Dependent Reaction Diffusion Model

A reaction-diffusion model can be used to model both the spatial and temporal changes in the electrospun fibers upon gas exposure. With the assumption that the reaction is reversible and first order with respect to each of its reactants and products, the concentration changes can be described generically by the following system of partial differential equations:

$$\begin{matrix}\frac{\partial Θ}{\partial τ} = \frac{1}{r} \frac{\partial }{\partial r} ( r \frac{\partial Θ}{\partial r} ) + α [ - Da ΘΦ + \frac{Da}{K} ΩΨ ] & ( 5 ) \\ \frac{\partial Φ}{\partial τ} = - Da ΘΦ + \frac{Da}{K} ΩΨ & ( 6 ) \\ \frac{\partial Ω}{\partial τ} = α [ Da ΘΦ - \frac{Da}{K} ΩΨ ] & ( 7 ) \\ \frac{\partial Ψ}{\partial τ} = Da ΘΦ - \frac{Da}{K} ΩΨ & ( 8 )\end{matrix}$$

Here, Θ is the normalized concentration of the diffusing gaseous reactant (e.g., NH₃ or NO₂), Φ is the normalized concentration of the non-diffusing reactant (e.g., PAni or PAni−H⁺), Ψ is the normalized concentration of the polymeric product of reaction (e.g., PAni−H⁺ or PAni), and Ω is the normalized concentration of the other product of reaction (e.g., NH₄⁺ or NO₂⁻). r is the normalized radius r/L. τ is the dimensionless time τ=t/t_{D}=tD/L² with D being the diffusivity of the diffusing gaseous reactant within the fiber. Da is the Damköhler number, defined as the dimensionless number representing the ratio of the reaction time constant, with respect to the forward reaction and reference concentration of Θ, to the diffusion time constant, so that Da=k_{f}C_{0,Θ}L²/D. K is the equilibrium constant of the reaction, also the ratio of the forward to reverse reaction rate constants K=k_{f}/kᵣ. α is the dimensionless ratio of the reference concentrations for the non-diffusing and diffusing reactant: α=C_{0,Φ}/C_{0,Θ}. Because of their corresponding stoichiometric ratios, the reference concentration of Ψ, C_{o,Ψ}, is set equal to the reference concentration of Φ, and the reference concentration of Ω, C_{o,Ω}, is set equal to the reference concentration of Θ. Equation 5 expresses the dynamics for the concentration of the gaseous reactant, which include diffusion down a concentration gradient, consumption by the forward reaction and production by the reverse reaction. Equation 7 expresses the dynamics for the concentration of the product formed from the gaseous reactant; as the product is generally ionic and believed to bind closely with the oppositely charged ions on the polymeric substrate, the dynamics do not include diffusion (assumed to be negligible), but include only production by the forward reaction and consumption by the reverse reaction. Equation 6 (or 8) expresses the dynamics for the concentration of the polymeric reactant (product), which also include only consumption (production) by the forward reaction and production (consumption) by the reverse reaction.

With the specification of appropriate boundary and initial conditions, this system can be solved numerically by MATLAB for specified values of the parameters. If one assumes that only the non-diffusing polymeric reactant is present in the system initially, and the initial concentrations of solute species (both reactant and product) within the fiber are zero, the boundary and initial conditions for the cylindrical system can be described as follows:

$$Θ ( 1 , τ ) = 1 , \frac{\partial Θ}{\partial r} ( 0 , τ ) = 0 , Θ ( r , 0 ) = 0$$

$$Φ ( r , 0 ) = 1$$

$$Ω ( r , 0 ) = 0$$

$$Ψ ( r , 0 ) = 0$$

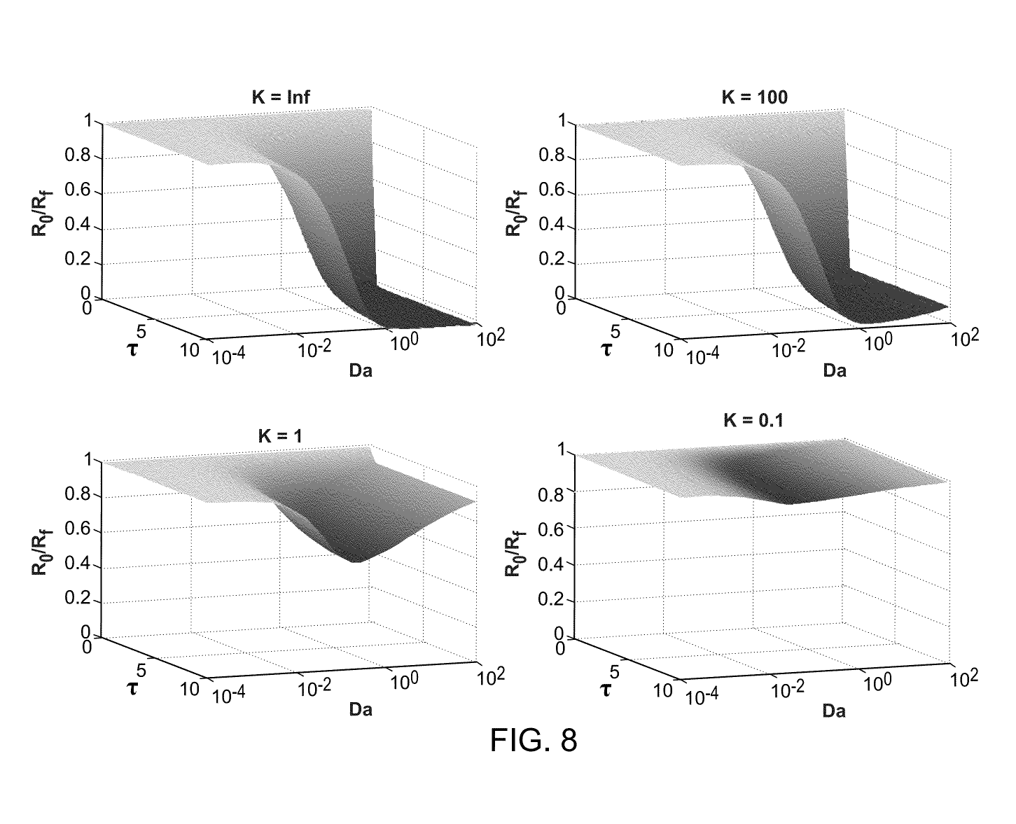

FIG. 8 shows the results of this reaction-diffusion model, where the ratio of initial to final resistances is plotted as a function of Da and τ at selected values of K=∞, 100, 1, and 0.1. For these calculations, we assume that the initial concentrations of small molecular solute species are zero, and that the conductivity of a section of the fiber decreases linearly with the concentration of the reactant Φ.

One can see that the resistance increases (R₀/Rₑₓ decreases) monotonically with τ for all values of Da and K. If Da is very large, the reaction is much faster than the diffusion; the diffusion front is very sharp but penetrates slowly into the fibers. If Da is very small, diffusion is much faster than reaction, so that the concentration profile is almost flat within the fiber; the gas rapidly penetrates the entire fiber. However, it may still take a long time (on the dimensionless scale) for the diffused gas to react and cause the necessary change in conductivity. Significantly, there exists a minimum in R₀/Rₑₓ with respect to Da at any given τ, except for the case of K=∞ where the forward reaction is irreversible. Therefore, there exists an optimal Da value for the overall resistance of the fiber to change at the fastest rate. This suggests that systems can be optimized with respect to Da for all such reversible reactions. Recalling that Da=k_{f}C_{0,Θ}L²/D, such optimization can be performed for a specific application by designing the fiber diameter for the target exposure concentration. Indirectly, Da can also be altered by changing the fiber material, gas species, or temperature, factors that all affect the reaction rate constant and diffusivity.

Equilibrium Determination from Steady-State Data

At steady state, where there is no longer dependence on time, the system of equations simplifies to:

$$\begin{matrix}\frac{\partial Θ}{\partial τ} = \frac{1}{r} \frac{\partial }{\partial r} ( r \frac{\partial Θ}{\partial r} ) = 0 & ( {5}^{′} ) \\ \frac{\partial Φ}{\partial τ} = - Da ΘΦ + \frac{Da}{K} ΩΨ = 0 & ( {6}^{′} ) \\ \frac{\partial Ω}{\partial τ} = α [ Da ΘΦ - \frac{Da}{K} ΩΨ ] = 0 & ( {7}^{′} ) \\ \frac{\partial Ψ}{\partial τ} = Da ΘΦ - \frac{Da}{K} ΩΨ = 0 & ( {8}^{′} )\end{matrix}$$

where equations (6′), (7′) and (8′) all reduce to the definition of the equilibrium constant being the ratio of the four concentrations at equilibrium, and equation (5′) requires that the radial dependence of concentration disappears at steady state. Since the concentrations, and thus the fiber electrical conductivity, are no longer dependent on radial position in the fiber, this leads to the simplification in equation 4 that

$$\frac{{R}_{ex}}{{R}_{0}} = \frac{{σ}_{0}}{{σ}_{ex}} . \qquad ( {4}^{′} )$$

Equation (4′) can be used to re-plot the experimental data for ΔR/R vs. gas phase concentration (at steady state) as a relationship between conductivity after exposure σₑₓ and gas phase concentration.

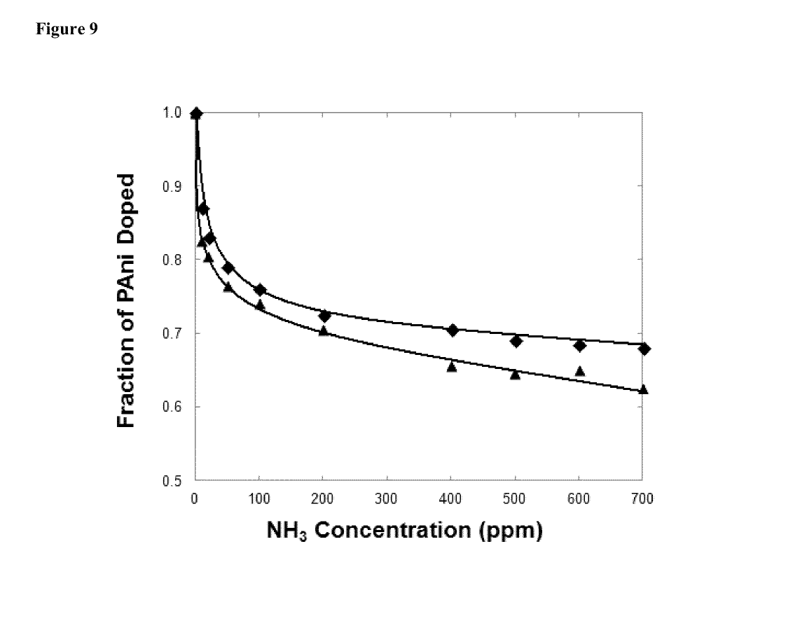

Take the system of ammonia sensing, for example. Once the experimental steady state data have been converted to a plot of σₑₓ vs. gas phase ammonia concentration, a plot such as the one given in FIG. 9, showing the relationship between fractions of PAni doped versus the external ammonia concentration, can be constructed. Here, we have used the results for conductivity vs [HCSA]/[PAni] shown in Table 1 as a calibration to relate conductivity to fraction of PAni doped.

By mass balance [PAni−H⁺]+[PAni]=[PAni]₀, where the right hand side is the original concentration of PAni present in the fibers, regardless of doping levels. Using this, the fraction of doped PAni can be related to the equilibrium constant, and written as

$$\frac{[ PAni - {H}^{+} ]}{{[ PAni ]}_{0}} = {( \frac{K [ {NH}_{3} ]}{[ {NH}_{4}^{+} ]} + 1 )}^{- 1} = 1 - \frac{K [ {NH}_{3} ]}{K [ {NH}_{3} ] + [ {NH}_{4}^{+} ]} \qquad ( 9 )$$

For each data point in FIG. 9, the fraction of PAni doped corresponds to a value of K[NH₃]ₛ/[NH₄⁺] according to Equation 9. Here, the subscript s is used to emphasize that these are concentrations of the ammonia and ammonium ion in the solid phase of the fiber, rather than the gas phase. The concentration of ammonia at the surface of the fiber is assumed to be in equilibrium with the exposed gas phase concentration of ammonia, with a partition coefficient, S, [NH₃]ₛ=S[NH₃]_{g} according to Henry's Law. At steady state, this same concentration of ammonia pervades the entire fiber. The concentrations of de-doped PAni and ammonium ion are both equal to the extent of reaction, ξ, as neither was present in the fiber initially, and neither species diffuses within the fiber. This allows the equilibrium constant K to be expressed in terms of the extent of reaction as follows

$$K = \frac{{[ {NH}_{4}^{+} ]}_{s} [ PAni ]}{{[ {NH}_{3} ]}_{s} [ PAni - {H}^{+} ]} = \frac{{ξ}^{2}}{{S [ {NH}_{3} ]}_{g} ( {[ PAni ]}_{o} - ξ )} \qquad ( 10 )$$

which is a quadratic equation in ξ

ξ²+SK[NH₃]_{g}ξ−SK[NH₃]_{g}[PAni]ₒ=0 (11)

The non-negative root of the equation is thus

$$ξ = \frac{\sqrt{{{( SK )}^{2} [ {NH}_{3} ]}_{g}^{2} + 4 {{SK [ {NH}_{3} ]}_{g} [ PAni ]}_{o}} - {SK [ {NH}_{3} ]}_{g}}{2} \qquad ( 12 )$$

The values of external ammonia concentrations are known. For the as-spun PAni fibers, [PAni]₀=5.0×10³ mol/m³, based on the known fiber density value of 1.0 g/cm³. Assuming a value for SK, we solve for the theoretical extent of reaction corresponding to each gas concentration, and obtain theoretical values for K[NH₃]ₛ/[NH₄⁺]ₛ=ξ/([Pani]₀−ξ) at each concentration. A least-squared residual analysis is then performed on the difference between experimental and theoretical values of (K[NH₃]ₛ/[NH₄⁺]ₛ) to find the best fit for value of SK.

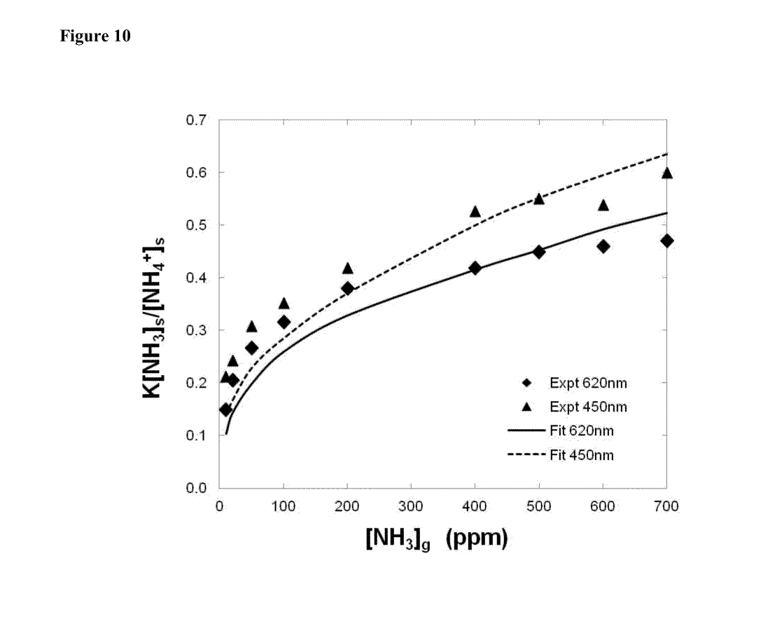

From the experimental steady-state data for the ammonia sensing by as-spun fibers (620 nm in diameter), the value of the equilibrium constant SK was thus determined to be 1.5±0.1. Using this value, the theoretical values are plotted in FIG. 10 as the solid curve.

As can be seen from FIG. 10, the experimental results for equilibrium extent of reaction for the 620 nm fibers and the 450 nm are systematically different over the whole range of external gas concentrations. We speculate that the solid state drawing process used to produce the smaller diameter fibers results in small but significant changes in the morphology, which may be reflected by subtle changes in S and/or [PAni]₀. For purpose of the present analysis, we held constant the value of SK=1.5 obtained for the as-spun fibers and varied [PAni]₀ in order to obtain the least squares residual for the data from the solid-state drawn fibers (450 nm diameter), The value obtained was [PAni]₀=3.8×10³ mol/m³. The theoretical curve using these values is shown in FIG. 10 as the dotted line.

In order to separate the estimate of SK into values for S and K, respectively, the results of the QCM analysis were used to determine the change in mass of the electrospun fiber sample (d=620 nm) before and after exposure to various concentrations of NH₃ in nitrogen. Assuming that the change in mass is due entirely to uptake of ammonia, a portion of which is converted to ammonium ion, change in mass can be expressed by Equation 13,

$$\frac{Δ m}{{m}_{o}} = \frac{{S [ {NH}_{3} ]}_{g} + ξ}{{[ PAni ]}_{o}} . \qquad ( 13 )$$

where m₀ is the original mass of the fibers obtained from the change in frequency due to deposition of fibers before exposure to gas, Δm is the change in mass calculated from frequency shifts due to gas exposure. From the expression for ξ in Equation 12, and known values of SK, gas concentrations and PAni concentrations, Table 4 lists the value of S determined at each of the three gas concentrations tested; the average gives S=0.05±0.01. The value of the equilibrium constant is then determined to be K=30±8.

TABLE 4
Calculation of Partition Coefficient, S, of ammonia in as-electrospun
PAni fibers (d = 620 nm)

| [NH₃]_{g} | | ξ | |
|---|---|---|---|
| (ppm) | Δm/m₀ | (mol/m³) | S |
| 100 | 0.098 ± 0.002 | 500 ± 60 | 0.045 ± 0.005 |
| 500 | 0.206 ± 0.002 | 1100 ± 80 | 0.055 ± 0.005 |
| 700 | 0.239 ± 0.003 | 1200 ± 90 | 0.049 ± 0.008 |

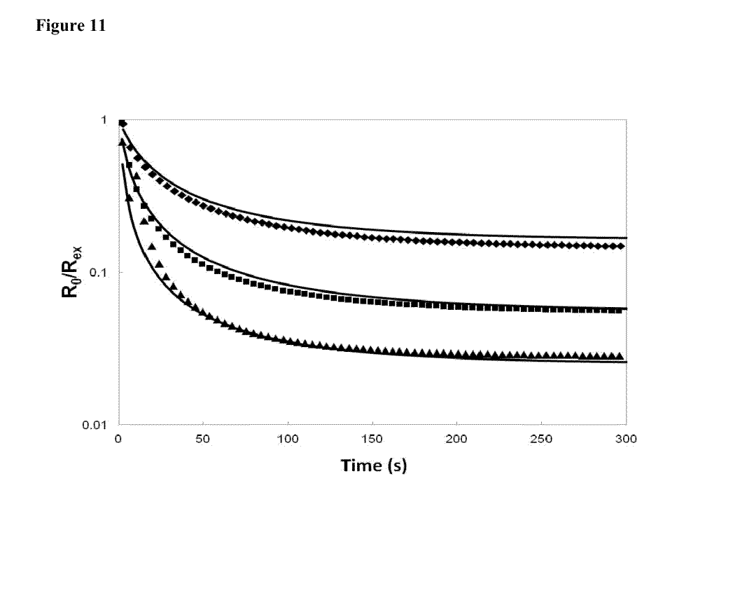

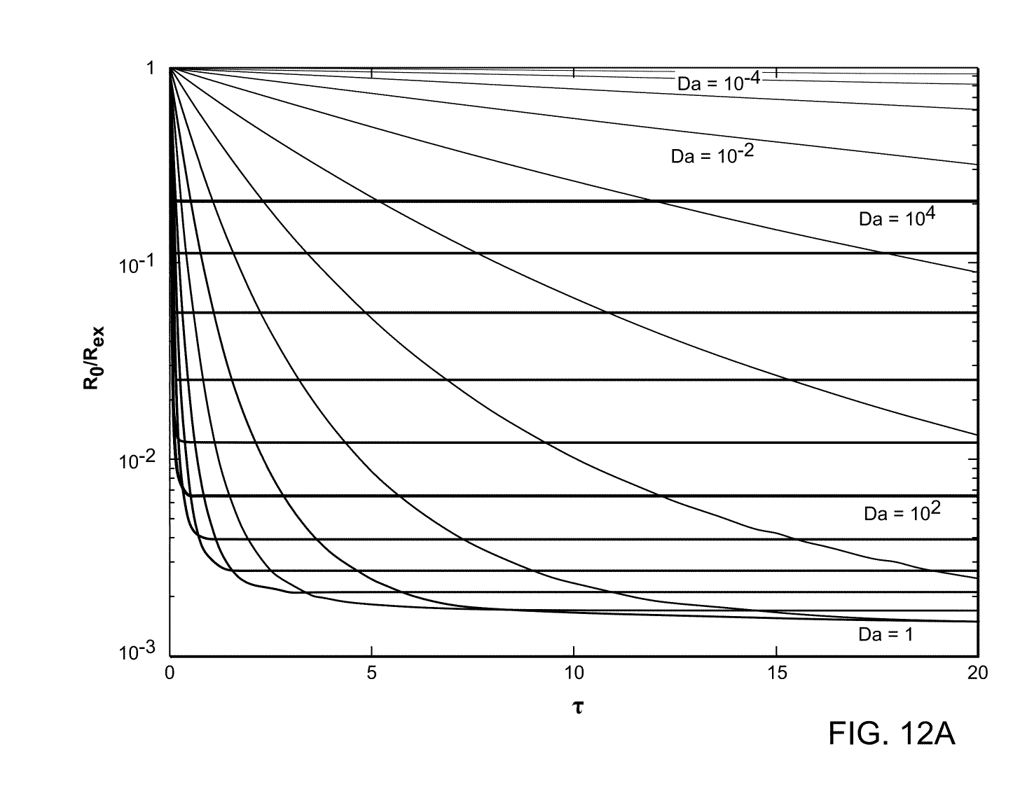

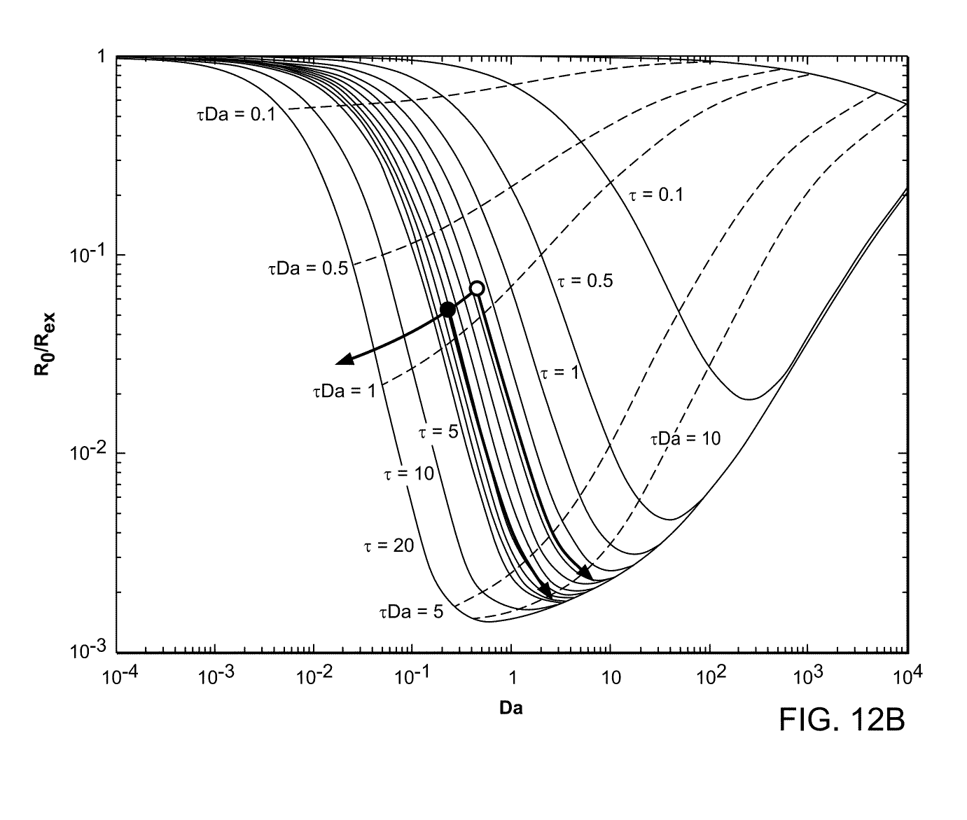

Having determined values for S and K, henceforth for purposes of dynamical analysis, the reference concentration for Θ is the solid phase concentration of ammonia at the surface of the fiber (r=1), which is related to the gas phase concentration of ammonia by the partition coefficient, S; that is, c_{o,Θ}=[NH₃]_{s,r=1}=S[NH₃]_{g} according to Henry's Law. Assuming that the reaction rate constants, diffusivity, and partition coefficient are not functions of gas concentration or fiber diameter, and that the gas phase is well mixed, the ratio (k_{f}/D)=Da/S[NH₃]_{g} L² is a parameter that can be determined by fitting to dynamical data such as that shown in FIG. 4. The other variable in the model is τ=tD/L², where real time t and fiber diameter L are known. We then fit the time-dependent sensing data with the modeled values to obtain estimates for k_{f} and D. FIG. 12 shows the reaction-diffusion model results for K=30 and S=0.05. A least-squares residual fitting using all of the time-dependent data for as-spun fibers (620 nm diameter) measured with gaseous ammonia concentrations from 10 ppm to 500 ppm gives the diffusivity D=(3.0±0.5)×₁₀⁻¹¹ cm²/s, and the forward rate constant k_{f}=0.15±0.07 cm³/mol/s. The comparison between experimental data and fitted values is shown in FIG. 11. These values are in reasonable agreement with reported literature values, where the diffusion coefficient of ammonia in polymer is on the order of 10⁻¹¹ to 10⁻⁹ cm²/s, and k_{f} is on the order of 0.001 to 0.1 cm³/mol/s.

TABLE 5
Summary of Values for Fitted Model Parameters

| Fitted Parameter | Value |
|---|---|
| SK | 1.5 ± 0.1 |
| S | 0.05 ± 0.01 |
| K | 30 ± 8 |
| D | (3.0 ± 0.5) × 10⁻¹¹ cm²/s |
| k_{f} | 0.15 ± 0.07 cm³/(mol s) |

Application of Model for Design Optimization

For optimization purposes, we assume that detection is required within a predetermined time, e.g., t=60 s. For the 450 nm diameter PAni fibers at 500 ppm of ammonia exposure in air at a density of approximately 1 kg/m³, and the values provided in Table 5, we obtain τ=3.6 and Da=0.23. From the model plots in FIG. 12, we find the optimal condition for this value of τ to be Da=2.3 and R₀/Rₑₓ=0.0020. That is, for the 450 nm PAni fibers studied here, the optimal detection at 60 s corresponds to a ΔR/R₀ of about 500, obtained under 5000 ppm of NH₃ exposure:

$$\begin{matrix} {( \frac{Δ R}{{R}_{0}} )}_{opt , t = 3.6} = {[ {( \frac{{R}_{0}}{{R}_{ex}} )}_{opt} ]}^{- 1} - 1 = 500 & ( 14 ) \\ \frac{{Da}_{opt}}{{Da}_{ref}} = \frac{{( {[ {NH}_{3} ]}_{g} )}_{opt}}{{( {[ {NH}_{3} ]}_{g} )}_{ref}} ⇒ {( {[ {NH}_{3} ]}_{g} )}_{opt} = \frac{2.3}{0.23} \times 500 ppm = 5000 ppm & ( 15 )\end{matrix}$$

For the same detection time of t=60 s and L=620 nm, τ=1.9 and the optimal values are Da=5.5 and R₀/Rₑₓ=0.0025, corresponding to a ΔR/R₀ ratio for PAni of this fiber diameter (620 nm) of 390 at 6200 ppm of NH₃ exposure.

Next, we optimize fiber diameter for a given detection time and gas exposure. In this case, both τ and Da vary with L, so the optimization trajectory does not follow a single contour line shown in FIG. 12. Instead, the product, τDa=tk_{f}S[NH₃]_{g} is constant. For a detection time of 60 s and exposure to a concentration of 500 ppm, the best sensing results are obtained at Da=0.016 and τ=52, with R₀/Rₑₓ=0.029. This condition corresponds to a fiber diameter of 60 nm, and is expected to show a ratio of resistance change of ΔR/R₀=36 after 60 s of exposure.

### INCORPORATION BY REFERENCE

All of the U.S. patents and U.S. published patent applications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A sensor comprising a plurality of fibers, wherein the plurality of fibers is configured as a non-woven material; the fiber consists essentially of (i) a polymer, or (ii) a polymer and a dopant; the polymer is selected from the group consisting of: a polyacetylene, a polypyrrole, a polythiophene, and a poly(3,4-ethylenedioxythiophene) (PEDOT); and the fiber has at least one dimension of about 1 nm to about 1 μm.

2. The sensor of claim 1, wherein the fiber consists essentially of a dopant and a polymer.

3. The sensor of claim 2, wherein the dopant is selected form the group consisting of HCSA, HCl, HClO₄, HI, FeCl₃, 4-dodecylbenzenesulfonic acid, p-toluenesulfonic acid, and dinonylnaphthalenedisulfonic acid.

4. The sensor of claim 1, wherein the polymer is selected from the group consisting of: polyacetylene, polypyrrole, polythiophene, and poly(3,4-ethylenedioxythiophene) (PEDOT).

5. A method of detecting a gas in a sample, comprising the steps of:
optionally determining the electrical resistance (R₀) or electrical conductance of a sensor;
contacting with the sensor a quantity of the sample; and
after a period of time, determining the electrical resistance (Rₑₓ) or electrical conductance of the sensor,
wherein the sensor comprises a plurality of fibers; the plurality of fibers is configured as a non-woven material; the fiber consists essentially of (i) a polymer, or (ii) a polymer and a dopant; the polymer is an intrinsically conducting polymer; and the fiber has at least one dimension of about 1 nm to about 1 μm.

6. The method of claim 5, wherein the gas is an oxidizing gas.

7. The method of claim 5, wherein the gas is an oxidizing gas selected from the group consisting of: NO₂, HCl, CO₂, O₃, H₂S, and SO₂.

8. The method of claim 5, wherein the gas is a reducing gas.

9. The method of claim 5, wherein the gas is a reducing gas selected from the group consisting of: NH₃, H₂, NO, and CO.

10. The method of claim 5, wherein the fiber consists essentially of a polymer selected from the group consisting of: a polyaniline, a polyacetylene, a polypyrrole, a polythiophene, and a poly(3,4-ethylenedioxythiophene) (PEDOT).

11. The method of claim 5, wherein the fiber consists essentially of a polymer selected from the group consisting of: a polyaniline, a polyacetylene, a polypyrrole, a polythiophene, and a poly(3,4-ethylenedioxythiophene) (PEDOT); and the gas is an oxidizing gas.

12. The method of claim 5, wherein the fiber consists essentially of a dopant and a polymer; and the polymer is selected from the group consisting of: a polyaniline, a polyacetylene, a polypyrrole, a polythiophene, and a poly(3,4-ethylenedioxythiophene) (PEDOT).

13. The method of claim 5, wherein the fiber consists essentially of a dopant and a polymer; and the polymer is selected from the group consisting of: a polyaniline, a polyacetylene, a polypyrrole, a polythiophene, and a poly(3,4-ethylenedioxythiophene) (PEDOT); and the gas is a reducing gas.

14. A method of detecting and quantifying a gas in a sample, comprising the steps of:
(a) optionally determining the electrical resistance (R₀) or electrical conductance of a sensor of claim 1;
(b) contacting with the sensor a first standard sample, wherein the concentration of the gas in the first standard sample is known;
(c) after a period of time, determining the electrical resistance or electrical conductance of the sensor;
(d) contacting with the sensor a second standard sample, wherein the concentration of the gas in the second standard sample is known; and the concentration of the gas in the second standard sample is different from the concentration of the gas in the first standard sample;
(e) after a period of time, determining the electrical resistance or electrical conductance of the fiber;
(f) contacting with the sensor a quantity of the sample; and
(g) after a period of time, determining the electrical resistance (Rₑₓ) or electrical conductance of the sensor,
wherein the sensor comprises a plurality of fibers; the plurality of fibers is configured as a non-woven material; each fiber consists essentially of (i) a polymer, or (ii) a polymer and a dopant; the polymer is an intrinsically conducting polymer; and each fiber has at least one dimension of about 1 nm to about 1 μm.

15. The method of claim 14, wherein the gas is an oxidizing gas.

16. The method of claim 14, wherein the gas is an oxidizing gas selected from the group consisting of: NO₂, HCl, CO₂, O₃, H₂S, and SO₂.

17. The method of claim 14, wherein the gas is a reducing gas.

18. The method of claim 14, wherein the gas is a reducing gas selected from the group consisting of: NH₃, H₂, NO, and CO.

19. The method of claim 14, wherein the fiber consists essentially of a polymer selected from the group consisting of: a polyaniline, a polyacetylene, a polypyrrole, a polythiophene, and a poly(3,4-ethylenedioxythiophene) (PEDOT).

20. The method of claim 14, wherein the fiber consists essentially of a polymer selected from the group consisting of: a polyaniline, a polyacetylene, a polypyrrole, a polythiophene, and a poly(3,4-ethylenedioxythiophene) (PEDOT); and the gas is an oxidizing gas.

21. The method of claim 14, wherein the fiber consists essentially of a dopant and a polymer; and the polymer is selected from the group consisting of: a polyaniline, a polyacetylene, a polypyrrole, a polythiophene, and a poly(3,4-ethylenedioxythiophene) (PEDOT).

22. The method of claim 14, wherein the fiber consists essentially of a dopant and a polymer; and the polymer is selected from the group consisting of: a polyaniline, a polyacetylene, a polypyrrole, a polythiophene, and a poly(3,4-ethylenedioxythiophene) (PEDOT); and the gas is a reducing gas.
